(19) United States
(12) Patent Application Publication
(10) Pub. No.: US 2024/0384351 A1
(43) Pub. Date: Nov. 21, 2024

(54) METHOD FOR CHARACTERIZING A TUMOR USING TARGETED SEQUENCING

(71) Applicant: ONCODNA, Gosselies (BE)

(72) Inventors: Jean-François LAES, Nivelles (BE); Maxime LIENARD, Auvelais (BE); Rim ZAAG, Evry (BE)

(21) Appl. No.: 18/547,872

(22) PCT Filed: Feb. 24, 2022
(86) PCT No.: PCT/EP2022/054710

§ 371 Date: Aug. 1, 2024

(30) Foreign Application Priority Data
Feb. 25, 2021 (BE) BE2021/5136

(51) Int. Cl. C12Q 1/6886

(52) U.S. Cl. CPC C12Q 1/6886; C12Q 2600/156; C12Q 2600/158

(57) ABSTRACT

A method for characterizing a tumor by targeted sequencing includes harvesting genomic DNA from a tumor, sequencing DNA fragments and characterizing the tumor. The sequencing and characterization include hybridizing DNA fragments using a plurality of double-stranded DNA hybridization probes to give hybridized fragments and uniformly enriching simple and complex DNA sequences of the hybridized DNA fragments. The tumor characterization is defined by sequencing target DNA sequences so as to identify genetic aberrations in the tumor.

### CROSS-REFERENCE TO RELATED APPLICATIONS

The present application is a National Stage Filing of PCT International Application No. PCT/EP2022/054710 filed on Feb. 24, 2022, which claims priority to Belgian application No. BE2021/5136, filed with the Belgian Patent Office on Feb. 25, 2021, which applications are incorporated herein by reference in their entirety.

### TECHNICAL FIELD

The present invention relates to a method for characterising a tumour by targeted sequencing from a sample from a cancer patient, comprising the steps of:
harvesting genomic DNA from said sample comprising at least one cancer cell,
preparing a sequencing library from said sample comprising the successive steps of (i) fragmenting said genomic DNA, generating DNA fragments, (ii) adding a series of tags to the ends of said DNA fragments, (iii) a first amplification of said DNA fragments using primers having a sequence complementary to a sequence of said tags forming a first mixture comprising amplified DNA fragments,
blocking said amplified DNA fragments of said first mixture using a series of blockers having a sequence complementary to at least one portion of the sequence of tags to form blocked DNA fragments, and
sequencing and characterising the tumour.

A medical condition in a patient is generally treated with a treatment regimen or therapy selected according to clinical criteria; i.e., a therapy or a treatment is selected for a patient based on a determination that the patient has been diagnosed with a particular illness (diagnosis has been established from conventional diagnostic tests). Although the molecular mechanisms underlying various medical conditions, in particular various cancers, have been studied for years, specifically using the molecular profile of a sick individual to determine a therapeutic regimen and targeted therapy for that individual has not been widely pursued.

Cancer is a disease characterised by multiple genetic aberrations that may be different depending on the type of cancer, and that may even be very different for the same type of cancer in different patients. For the purposes of the present invention, the term “type of cancer” means cancer that affects the same tissue or organ.

Without being exhaustive, a genetic aberration may comprise a nucleotide point mutation, a single nucleotide polymorphism, an insertion, a deletion, a translocation, a variation in copy number of a gene or a specific DNA sequence, a deletion of one or two copies of a gene, a homologous recombination deficiency (HRD) phenotype, a specific tumour mutational burden, a microsatellite instability or even an allelic imbalance of telomeres. The tumour may be characterised by one or more different genetic aberrations. From one patient to another, given that there may be a huge amount of mutational heterogeneity for the same type of cancer, the efficacy of a cancer treatment may be very different depending on the patient. A cancer treatment is generally specific to certain molecular targets expressed in the tumour. For a cancer treatment to be effective, these molecular targets must be well expressed in the tumour. A tumour-specific mutational profile leads to certain cell signalling pathways and/or certain metabolic pathways being activated and/or inhibited, which may or may not result in the tumour expressing the specific molecular targets of the cancer treatment. As a result, the tumour may be sensitive or resistant to said cancer treatment. Therefore, it is important to be able to specifically identify the molecular profile and the genetic profile of each tumour as accurately as possible, so as to be able to offer the patient a treatment that is best suited to their molecular and genetic profile. In addition to the genetic profile of the tumour determined by identifying genetic aberrations, for the purposes of the present invention, it is to be understood that the molecular profile of the tumour means determining the expression of molecular markers of the tumour, which may comprise RNAs and/or microRNAs and/or proteins.

Targeted sequencing is a sequencing technique that enables genomic regions of interest to be specifically selected prior to the sequencing step. Targeted sequencing avoids having to sequence the whole genome (whole genome sequencing) or the whole exome (whole exome sequencing). In order to carry out targeted sequencing of specific genomic regions, these genomic regions must be captured and enriched in a specific way before being sequenced.

### PRIOR ART

A method for characterising a tumour by targeted sequencing from a sample from a cancer patient is known, for example, from the document EP2721181, which describes a targeted sequencing method to characterise a tumour. According to this document, the hybridisation probes are conventional probes, which limits the number of tumour characteristics that can be identified given the number of false positives and false negatives generated by this type of targeted sequencing method. Conventional hybridisation probes will enrich certain DNA sequences of the tumour less effectively than others, which limits the amount of information generated with a given sequencing coverage and which generates false negatives and false positives when identifying genetic aberrations.

Document EP2981624 relates to targeted sequencing methods involved in evaluating samples (for example, cancer cells or nucleic acids derived therefrom) for a homologous recombination deficiency (HRD) phenotype (for example, HRD signature) based on the detection of particular chromosomal aberrations (CA). A HRD phenotype is characterised by a deficiency in the mechanism for repairing DNA double-strand breaks by homologous recombination. The document provides methods and materials to detect CA regions in order to determine whether a cell (for example, a cancer cell) has a HRD phenotype. Therefore, a cell with a HRD phenotype will be more sensitive to treatments causing DNA breaks. This document also provides materials and methods for identifying cancer patients who are likely to respond to a particular cancer treatment regimen based on the presence, absence or severity of a HRD.

Document EP2659005 relates to methods and a system for molecular profiling, including by targeted sequencing, using the results from molecular profiling to identify targeted treatments for individuals. The method for identifying a candidate treatment for a subject in need thereof comprises: (a) determining a molecular profile for one or more samples from the subject using a panel of genes or gene products, (b) comparing the molecular profile of the subject to a reference molecular profile to identify which panel members are expressed differently between the sample(s) and the reference; and (c) identifying a treatment which is associated with one or more panel members, wherein for this purpose said one or more panel members are expressed differently between the sample(s) and the reference.

Document US2020118644 relates to the use of next-generation sequencing to determine microsstellite instability (MSI) status. This document presents techniques to determine microsatellite instability directly from maps of microsatellite regions for specific loci in the genome. The techniques provide an automated process for MSI testing and for predicting MSI status via a supervised machine learning process.

Unfortunately, such methods as described above still have numerous disadvantages. There is a growing demand for reliable identification of a large number of genetic aberrations in order to improve tumour characterisation. To date, in order to identify a large number of tumour characteristics using sequencing, the prior art suggests using whole genome sequencing which, by definition, provides an overall view of the genetic aberrations of a tumour but which is still too costly and too slow to be used for everyday medical diagnosis. An alternative to whole genome sequencing is to carry out targeted sequencing of certain predetermined genomic regions. However, to date, this type of targeted sequencing does not allow sufficient genetic aberrations of a tumour to be identified to accurately reflect the genetic complexity of a tumour sample with acceptable certainty. Conventional targeted sequencing techniques sequence certain genomic regions of the tumour less effectively than others, which makes the sequencing non-uniform and limits the amount of information generated with a given sequencing coverage. The above-mentioned non-uniformity of the sequencing and the limited amount of sequencing information generated with a given sequencing coverage additionally create a large number of false positives and false negatives when identifying genetic aberrations, which only allows the tumour to be characterised reliably with an insufficient number of genetic aberrations. If the amount of generated sequencing information is limited by the non-uniformity of the sequencing, it will result in low statistical power when identifying genetic aberrations, which leads to a significant number of false positives and false negatives. Reliably characterising a sufficient number of genetic aberrations of the tumour is important to establish the best-suited treatment for the patient. Given the intensity of cancer treatments for patients, for example in chemotherapy, immunotherapy, etc., in terms of both treatment duration and side effects, it is very important to avoid administering treatments without a response. In oncology, there are still numerous cases where the physician administers a treatment and, depending on the response of the patient, decides whether to continue treatment or to administer another therapy. However, such treatments have far-reaching consequences for the general health of the patient and when they have no response, precious time is sometimes lost. Also, recommending a chemotherapy or immunotherapy treatment identified on the basis of a false positive or a false negative may also be very detrimental to the patient. There is therefore a need to provide reliable methods for characterising the tumour in which the number of false positives and false negatives in the targeted sequencing of a tumour is kept to a minimum while still being accessible and capable of being carried out within an acceptable timeframe.

### SUMMARY OF THE INVENTION

The present invention aims to reduce these disadvantages by providing a cheaper, faster and more reliable method compared with conventional tumour characterisation techniques. For this, the present invention provides a method for characterising a tumour by targeted sequencing from a sample from a cancer patient wherein said sequencing and said tumour characterisation comprise the steps of:
collecting a plurality of double-stranded DNA hybridisation probes specific to a plurality of said blocked DNA fragments,
denaturing said double-stranded DNA hybridisation probes to form a plurality of denatured DNA hybridisation probes specific to said plurality of said blocked DNA fragments,
hybridising the plurality of said blocked DNA fragments by the plurality of denatured DNA hybridisation probes to form a second mixture comprising DNA fragments hybridised and DNA fragments not hybridised to the plurality of denatured DNA hybridisation probes,
uniformly and simultaneously enriching the second mixture with DNA fragments containing simple sequences and with DNA fragments containing complex sequences of said DNA fragments hybridised to the plurality of denatured DNA hybridisation probes by capturing said hybridised DNA fragments to form a medium enriched with said DNA fragments hybridised to the plurality of denatured DNA hybridisation probes,
washing and recovering said DNA fragments hybridised to the plurality of denatured DNA hybridisation probes to form a medium enriched with target DNA sequences,
a second amplification of said target DNA sequences using primers having a sequence complementary to a sequence of said tags, giving target DNA sequences to be sequenced,
said tumour characterisation being characterising by sequencing DNA fragments containing simple sequences and DNA fragments containing complex sequences of said target DNA sequences to be sequenced so as to identify genetic aberrations in the tumour.

According to the present invention, the specific double-stranded DNA hybridisation probes uniformly enrich both DNA fragments containing simple sequences and DNA fragments containing complex sequences because when the specific double-stranded DNA hybridisation probes are denatured, the two DNA strands of each specific double-stranded DNA hybridisation probe (denatured DNA hybridisation probes) hybridise to the sense strand of the target genomic region and the antisense strand to said sense strand of the target genomic region. Hybridising denatured DNA hybridisation probes to the sense and antisense strand of the target genomic region enables more efficient capture, and therefore more uniform enrichment of different types of DNA fragment such as DNA fragments containing simple sequences and DNA fragments containing complex sequences. The targeted sequencing following this enrichment step then enables DNA fragments containing simple sequences and DNA fragments containing complex sequences to be sequenced much more uniformly because the enrichment is more uniform. This then enables more tumour characteristics to be identified, and ultimately ensures a more comprehensive tumour characterisation, at a reduced cost.

Having a uniform enrichment of simple and complex DNA sequences of said DNA fragments hybridised to the plurality of denatured DNA hybridisation probes enables uniform and optimal sequencing of the simple and complex sequences of said target DNA sequences to be sequenced. This uniform sequencing enables much more sequencing information to be generated for a given sequencing coverage. Generating as much sequencing information as possible for a given sequencing coverage limits the number of false positives and false negatives obtained when identifying genetic aberrations in the tumour because the identification of genetic aberrations will be based on a greater number of sequencing data and therefore on greater statistical power, which makes the method according to the present invention cheaper and more reliable.

Although techniques for enriching target genomic regions with double-stranded DNA hybridisation probes are known (for example, see documents WO2019/008172A1, US2014/243232A1, US2020017907A1), techniques based on double-stranded DNA hybridisation probes are sometimes not applied in the field of oncology to identify a wide range of genetic aberrations or not applied at all for non-coding and/or complex sequences. However, to identify a wide range of genetic aberrations in the tumour, it is necessary to be able to enrich and sequence complex genomic regions because these contain a large amount of information that enables these tumour genetic aberrations to be better identified and therefore a more comprehensive tumour characterisation to be provided. However, before these complex genomic regions can be enriched and sequenced, they must be identified in such a way that these complex genomic regions are representative of multiple genetic aberrations. However, tumour samples are characterised by a significant amount of necrotic tissue and/or apoptotic cells which reduce the quality of the extracted DNA and thus limit the sequencing possibilities thereof. Furthermore, because each tumour has a specific mutational profile, the sequence of genomic regions of a tumour may vary greatly depending on the tumour analysed. As a result, there was no reason to expect more uniform sequencing coverage using double-stranded DNA hybridisation probes to sequence both genomic regions containing simple sequences and complex sequences of the tumour. These genomic regions may have different mutational profiles depending on the tumour analysed and it is therefore surprising to be able to identify, with sufficient specificity and robustness, a plurality of tumour genetic aberrations providing a more comprehensive tumour characterisation, regardless of the type of tumour and tumour sample analysed.

For the purposes of the present invention, the term “specific double-stranded DNA hybridisation probes” refers to, for example, specific probes having a sequence complementary to a sequence selected from target genomic regions. For the purposes of the present invention, specific double-stranded DNA hybridisation probes therefore comprise at least one probe specific to a DNA fragment containing a simple sequence and at least one probe specific to a DNA fragment containing a complex sequence. Preferably, the specific double-stranded DNA hybridisation probes comprise at least 50, more particularly 100, advantageously at least 500 probes specific to one or more DNA fragments containing a simple sequence and at least 50, more particularly 100, advantageously at least 500 probes specific to one or more DNA fragments containing a complex sequence, even a total number of specific double-stranded DNA hybridisation probes between 100 and 20,000, more particularly between 200 and 10,000, favourably between 500 and 5,000.

For the purposes of the present invention, the terms “complex genomic region” or “complex sequences” refer to, for example,
regions rich in guanine and cytosine nucleotide bases as is often the case for promoter regions and the first exon of a gene, typically regions defined by nucleotide sequences having a percentage of guanine and cytosine base relative to the total number of nucleotide bases forming the nucleotide sequences equal to or greater than 60%, preferably equal to or greater than 70%, favourably equal to or greater than 80%, and/or
regions rich in adenine and thymine nucleotide bases, typically regions defined by sequences having a percentage of adenine and thymine base relative to the total number of nucleotide bases forming the nucleotide sequences equal to or greater than 60%, preferably equal to or greater than 70%, favourably equal to or greater than 80%, and/or
regions such as centromeres or telomeres, and/or
regions having a hairpin structure where one sequence contains two inverted nucleotide repeats which are separated by at least three nucleotides, and/or
repeat sequences defined by a repeat of two or three nucleotides.

As can be seen, the present invention therefore enables a tumour to be characterised by targeted sequencing from a sample from a cancer patient. The hybridisation and enrichment steps of targeted sequencing are carried out using a plurality of denatured DNA hybridisation probes derived from the denaturation of a plurality of double-stranded DNA hybridisation probes, which enables a more uniform enrichment of target DNA fragments containing simple sequences and target DNA fragments containing complex sequences. This uniform enrichment of all target DNA fragments enables all these DNA fragments to be sequenced very accurately, the number of false positives and false negatives to be reduced, a sufficient average sequencing coverage to be maintained and sequencing costs to be significantly reduced. The uniform sequencing of target DNA fragments of simple and complex sequences according to the present invention enables a large number of genetic aberrations of the tumour to be identified because numerous genetic aberrations are located in regions containing simple sequences, but also in complex genomic regions such as regions rich in guanine and cytosine nucleotide bases and/or centromeres and/or telomeres and/or regions rich in repeat sequences, ensuring a more comprehensive tumour characterisation.

According to the present invention, the term “primer having a sequence complementary to a sequence of said tags” means a primer that has a sequence having a sequence complementarity to a sequence of one or more of said tags of at least 80%, preferably at least 90%, even more preferably at least 92%, 93%, 94%, 95%, 96%, 97%, 98%, 99%, 100%.

According to the present invention, the term “blocker having a sequence complementary to at least one portion of the sequence of tags” means a blocker that has a sequence such that the sequence complementarity to the sequence of tags is at least 50%, preferably, at least 80%, more preferably at least 90%, advantageously at least 92%, 93%, 94%, 95%, 96%, 97%, 98%, 99%, 100%.

According to the present invention, the term “uniformly (or more uniformly) and simultaneously enriching the second mixture with DNA fragments containing simple sequences and with DNA fragments containing complex sequences of said DNA fragments hybridised to the plurality of denatured DNA hybridisation probes” means enriching simple and complex DNA sequences having an enrichment uniformity equal to or greater than 80%, more preferably equal to or greater than 85%, even more preferably equal to or greater than 90%, favourably equal to or greater than 91%, 92%, 93%, 94%, 95%, 96%, 97%, 98%, 99%. More particularly, enriching a first simple sequence will be, for example, similar to enriching a second complex sequence to within 20%, more preferably to within 15%, more particularly to within 10% or better still to within 9%, 8%, 7%, 6%, 5%, 4%, 3%, 2%, 1%.

More particularly, uniformly (or more uniformly) and simultaneously enriching simple and complex DNA sequences may also be defined as follows:
if the level of enrichment of the first simple sequence is X1, then the level of enrichment of the second complex sequence is Y1, such that Y1 is between 0.8*X1 and 1.2*X1, preferably between 0.85*X1 and 1.15*X1, more particularly between 0.9*X1 and 1.1*X1 or better still between 0.91*X1 and 1.09*X1, between 0.92*X1 and 1.08*X1, between 0.93*X1 and 1.07*X1, between 0.94*X1 1.06*X1, between 0.95*X1 and 1.05*X1, between 0.96*X1 and 1.04*X1, between 0.97*X1 and 1.03*X1, between 0.98*X1 and 1.02*X1, between 0.99*X1 and 1.01*X1, in such a way that the level of uniform and simultaneous enrichment of simple DNA sequences and complex DNA sequences X1/Y1 is between 0.8 and 1.2, preferably between 0.85 and 1.15, more particularly between 0.9 and 1.1, favourably between 0.91 and 1.09, between 0.92 and 1.08, between 0.93 and 1.07, between 0.94 and 1.06, between 0.95 and 1.05, between 0.96 and 1.04, between 0.97 and 1.03, between 0.98 and 1.02, between 0.99 and 1.01.

A given level of enrichment for each simple and/or complex DNA sequence is also understood to be equal to the arithmetic mean +/−20% of the level of enrichment of all simple and/or complex DNA sequences, more preferably, equal to the arithmetic mean +/−15%, or better still +/−10%, +/−9%, +/−8%, +/−7%, +/−6%, +/−5%, +/−4%, +/−3%, +/−2%, +/−1% of the level of enrichment of all simple and/or complex DNA sequences.

Advantageously, simple and complex DNA sequences are enriched simultaneously under the same conditions.

In addition, in the characterisation method according to the present invention, after hybridisation, washing and recovering said hybridised DNA fragments enables a large amount of impurities to be removed and therefore ensures better purity of the enriched medium comprising target DNA sequences, which enables a second optimal amplification of said target DNA sequences and therefore also optimal sequencing of said target DNA sequences to be sequenced.

Advantageously, in the characterisation method according to the present invention, the harvesting of genomic DNA from a sample comprising at least one cancer cell comprises one or more of the following steps:
receiving a sample composed of either a block comprising a formalin-fixed paraffin-embedded (FFPE block) piece of tumour extracted by biopsy, or tumour tissue slides already cut;
if the sample is composed of a block, cutting the block into slides with a thickness between 4 and 15 μm;
staining the first and last slide with hematoxylin and eosin (H&E slides);
identifying from the first slide, optionally by visual inspection, an area of the slide comprising sufficient tumour cells, little or no necrosis and lymphocytic infiltration of less than 21%;
marking a region comprising the most tumour cells on this first stained slide;
transferring the marking from the first slide to the unstained slides to mark the same tumour region, but on a different slice;
scraping the cells in these marked areas (macrodissection step);
extracting DNA from these slides to form the genomic DNA to be characterised;
in parallel, quantifying, using a spectrophotometer, DNA from a sample of genomic DNA to be characterised;
in parallel, qualifying DNA by gel migration from a sample of genomic DNA to be characterised.

Advantageously, in the characterisation method according to the present invention, said sample comprising at least one cancer cell is a blood sample or a urine sample. When tumour DNA is found in the form of DNA circulating in a blood sample or a urine sample, the characterisation method according to the present invention enables the tumour to be characterised without invasive intervention for the patient, while providing sufficient accuracy to characterise the genetic aberrations in the tumour.

Preferably, in the characterisation method according to the present invention, said DNA fragments containing complex sequences of said target DNA sequences to be sequenced being sequences with a percentage of guanine and cytosine nucleotide base equal to or greater than 60%, preferably equal to or greater than 70%, favourably equal to or greater than 80% or repetitive sequences or inverted sequences. These DNA fragments rich in guanine and cytosine are more difficult to denature and are more difficult to enrich during the enrichment step of targeted sequencing, especially as DNA fragments containing simple sequences and DNA fragments containing complex sequences are typically sequenced during the same step, i.e., under conditions that represent the best compromise for both simple sequences and complex sequences.

Advantageously, in the characterisation method according to the present invention, said DNA fragments hybridised to said plurality of denatured DNA hybridisation probes have a sequence of which at least a portion is a coding sequence of said DNA fragments containing simple sequences and/or said DNA fragments containing complex sequences and/or a sequence of which at least a portion is a non-coding sequence of said DNA fragments containing simple sequences and/or said DNA fragments containing complex sequences. For the purposes of the present invention, non-coding sequences are non-coding sequences of a gene or non-coding sequences outside the gene in relative proximity so that they are accessible for sequencing. The genetic aberrations that characterise a tumour are located at multiple positions in the genome within a coding or non-coding sequence of a gene or a sequence outside a gene. It is therefore advantageous to simultaneously target a set of DNA fragments representative of a plurality of possible genetic aberrations or an absence of possible genetic aberrations by hybridising these DNA fragments to a plurality of specific denatured DNA hybridisation probes, so as to characterise the tumour as comprehensively as possible. During the targeted sequencing of the present invention, DNA fragments containing simple sequences and DNA fragments containing complex sequences are sequenced simultaneously. The plurality of specific denatured DNA hybridisation probes, derived from specific double-stranded DNA hybridisation probes, target a set of DNA fragments that enable a plurality of possible genetic aberrations or an absence of genetic aberrations to be identified. This makes it possible to compensate for the heterogeneity of the mutational profile of one tumour to the next and to be extremely versatile because the genetic aberrations or the absence of genetic aberrations which are identified in the characterisation method according to the present invention may therefore be different from one type of tumour to the next and/or for different tumours of the same type of tumour.

Preferably, said target DNA sequences to be sequenced contain genetic aberrations comprising a series of single nucleotide polymorphisms of one or more DNA sequences relative to one or more corresponding DNA sequences of a reference genome, said series of single nucleotide polymorphisms preferably comprising at least 50, more preferably at least 500, even more preferably at least 1,500, favourably at least 3,000 single nucleotide polymorphisms, said single nucleotide polymorphisms being located, on average over the whole genome, every 0.5 to 50 megabases, preferably every 5 to 25 megabases, more preferably every 10 to 20 megabases, favourably every 14 to 15 megabases.

Advantageously, the reference genome is a human genome. Preferably, the nucleotide sequence of the reference genome is obtained using public databases such as hg19 (for example: grch37) from NCBI: https://www.ncbi.nlm.nih.gov/search/all/?term=grch37.p13. Advantageously, the reference genome may also be a genome from a healthy sample comprising healthy (non-tumour) cells from said cancer patient from whom said sample comprising at least one cancer cell has been taken. The genetic aberrations of the tumour of said patient may therefore be identified either by comparison with a reference genome such as the human genome from a public database such as hg19, or comparison with the genome from said healthy sample comprising healthy (non-tumour) cells from said cancer patient.

Preferably, in the method for characterising a tumour by targeted sequencing according to the present invention, said target DNA sequences to be sequenced contain genetic aberrations comprising a deletion of one or two copies of a gene relative to said reference genome. This genetic aberration enables, another among things, the loss of heterozygosity of certain genes to be determined, which is an important mechanism in tumour development, particularly when the loss of heterozygosity is present for tumour suppressor genes.

Advantageously, in the characterisation method according to the present invention, said target DNA sequences to be sequenced contain genetic aberrations comprising a variation in copy number relative to the copy number of said reference genome associated with at least 2 genomic regions, preferably at least 50 genomic regions, more preferably at least 100 genomic regions, favourably at least 250 genomic regions and at most 5,000 genomic regions, preferably at most 2,000 genomic regions, preferably at most 1,000 genomic regions randomly distributed in the genome. Identifying a variation in copy number associated with, for example, more than 250 genomic regions but less than 5,000 genomic regions randomly distributed in the genome provides a backbone for analysing variation in copy number that is representative of the tumour while limiting sequencing costs.

Advantageously, in the characterisation method according to the present invention, said target DNA sequences to be sequenced contain genetic aberrations comprising a homologous recombination deficiency phenotype determined by comparing at least one of said target DNA sequences to be sequenced with said at least one corresponding DNA sequence of said reference genome. A homologous recombination deficiency prevents normal repair of DNA damage, which may cause genetic aberrations such as deletions and/or duplications of genomic regions. It is therefore advantageous to determine whether the tumour analysed comprises a homologous recombination deficiency phenotype. In particular, the homologous recombination deficiency phenotype may be partly determined by the mutational status of the BRCA1 and BRCA2 genes which are involved in a normal DNA damage repair mechanism.

Preferably, in the characterisation method according to the present invention, said target DNA sequences to be sequenced contain genetic aberrations comprising a tumour mutational burden determined by comparing a plurality of coding sequences of said target DNA sequences to be sequenced with a plurality of corresponding coding sequences of said reference genome, said plurality of coding sequences of said target DNA sequences to be sequenced is determined by sequencing at least one megabase of coding sequences, preferably at least 1.1, more preferably at least 1.2, 1.3, 1.4, 1.5, 1.6, 1.7, 1.8, 1.9, 2 megabases of coding sequences of said target DNA sequences to be sequenced. Determining the tumour mutational burden of a large amount of coding sequence is advantageous because the more tumour mutational burden is determined by a large amount coding sequence, and by a large panel of genes, the more accurate the prediction of response to targeted therapies and immunotherapy will be.

Preferably, in the method for characterising a tumour by targeted sequencing according to the present invention, said target DNA sequences to be sequenced contain genetic aberrations comprising a microsatellite instability determined by comparing a series of said target DNA sequences to be sequenced with a series of corresponding DNA sequences of said reference genome, said series of said target DNA sequences to be sequenced is determined by sequencing at least one target DNA sequence to be sequenced, preferably at least 100 target DNA sequences to be sequenced, more preferably at least 1,000 target DNA sequences to be sequenced, favourably at least 1,500 target DNA sequences to be sequenced. Microsatellite instability is a process involved in tumour development which may lead to a high mutation rate in the tumour. Determining microsatellite instability may have predictive value in determining the efficacy of immunotherapy.

Advantageously, in the method for characterising a tumour by targeted sequencing according to the present invention, said tumour characterisation comprises sequencing at least one portion of a sequence of at least 100, more particularly at least 200, even more particularly at least 300, preferably at least 325, or better still, at least 350, 375, 400, 425, 450, 475, 500, 525, 550, 575, 600 genes, more particularly of each gene of a panel of genes listed in Table 1, or sequencing at least one portion of a sequence of each gene of a subgroup of genes included in Table 1 to identify a signature of genetic aberrations associated with a therapy. Sequencing the extended panel of genes listed in Table 1 enables the determination of genetic aberrations such as microsatellite instability and/or tumour mutational burden to be improved. The gene subgroups included in the panel of genes (group of genes) in Table 1, the sequencing of which enables the identification of genetic aberrations which are associated with mechanisms associated with a therapy (treatment), comprise: (i) a subgroup comprising the EGFR, MET and KRAS genes to identify treatments based on EGFR tyrosine kinase inhibitors in the context of lung cancer, more particularly non-small cell lung cancer (NSCLC), (ii) a subgroup comprising the EGFR, KRAS and BRAF genes to identify anti-EGFR therapies in the context of colorectal cancer (CRC), (iii) a subgroup comprising the BRCA1, BRCA2, PALB2 and ATM genes to identify treatments based on PARP inhibitors in the context of breast cancer or ovarian cancer, (iv) a subgroup comprising the APLNR, B2M, PD-L1, PD-L2 genes to identify treatments based on anti-PD1 or anti-PD-L1 inhibitors in the context of various cancers such as lung cancer, melanoma or bladder cancer.

TABLE 1
List of the panel of genes.

| | | | | | |
|---|---|---|---|---|---|
| ABL1 | ABL2 | ACVR1 | ACVR1B | AGO1 | AGO2 |
| AJUBA | AKT1 | AKT2 | AKT3 | ALB | ALK |
| ALOX12B | AMER1 | ANKRD11 | ANKRD26 | APC | APLNR |
| AR | ARAF | ARFRP1 | ARHGAP35 | ARID1A | ARID1B |
| ARID2 | ARID5B | ASXL1 | ASXL2 | ATM | ATR |
| ATRX | ATXN7 | AURKA | AURKB | AXIN1 | AXIN2 |
| AXL | B2M | BABAM1 | BAP1 | BARD1 | BAT25 |
| BAT-26 | BBC3 | BCL10 | BCL2 | BCL2L1 | BCL2L11 |
| BCL2L2 | BCL6 | BCOR | BCORL1 | BCR | BIRC3 |
| BLM | BMPR1A | BRAF | BRCA1 | BRCA2 | BRD4 |
| BRIP1 | BTG1 | BTG2 | BTK | C11orf30 | CALR |
| CARD11 | CARM1 | CASP8 | CBFB | CBL | CCNB3 |
| CCND1 | CCND2 | CCND3 | CCNE1 | CD276 | CD70 |
| CD74 | CD79A | CD79B | CDC42 | CDC73 | CDH1 |
| CDK12 | CDK4 | CDK6 | CDK7 | CDK8 | CDKN1A |
| CDKN1B | CDKN2A | CDKN2B | CDKN2C | CEBPA | CENPA |
| CHD2 | CHD4 | CHEK1 | CHEK2 | CIC | CMTR2 |
| CREBBP | CRKL | CRLF2 | CSDE1 | CSF1R | CSF3R |
| CSNK1A1 | CTCF | CTLA4 | CTNNA1 | CTNNB1 | CTR9 |
| CUL3 | CUL4A | CUX1 | CXCR4 | CYLD | CYP17A1 |
| CYP19A1 | CYP2C19 | CYP2D6 | CYSLTR2 | D2S123 | DAXX |
| DCUN1D1 | DDR1 | DDR2 | DDX41 | DHX15 | DICER1 |
| DIS3 | DNAJB1 | DNMT1 | DNMT3A | DNMT3B | DOT1L |
| DPYD | DROSHA | DUSP4 | E2F3 | EED | EGFL7 |
| EGFR | EIF1AX | EIF4A2 | EIF4E | ELF3 | EML4 |
| EMSY | EP300 | EPAS1 | EPCAM | EPHA3 | EPHA5 |
| EPHA7 | EPHB1 | EPHB4 | ERBB2 | ERBB3 | ERBB4 |
| ERCC1 | ERCC2 | ERCC3 | ERCC4 | ERCC5 | ERF |
| ERG | ERRFI1 | ESR1 | ETAA1 | ETS1 | ETV1 |
| ETV4 | ETV5 | ETV6 | EWSR1 | EZH1 | EZH2 |
| EZR | FAM175A | FAM46C | FAM58A | FANCA | FANCC |
| FANCD2 | FANCE | FANCF | FANCG | FANCI | FANCL |
| FAS | FAT1 | FBXW7 | FGF1 | FGF10 | FGF12 |
| FGF14 | FGF19 | FGF2 | FGF23 | FGF3 | FGF4 |
| FGF5 | FGF6 | FGF7 | FGF8 | FGF9 | FGFR1 |
| FGFR2 | FGFR3 | FGFR4 | FH | FLCN | FLI1 |
| FLT1 | FLT3 | FLT4 | FOXA1 | FOXF1 | FOXL2 |
| FOXO1 | FOXP1 | FRS2 | FUBP1 | FYN | GAB1 |
| GAB2 | GABRA6 | GATA1 | GATA2 | GATA3 | GATA4 |
| GATA6 | GEN1 | GID4 | GLI1 | GNA11 | GNA13 |
| GNAQ | GNAS | GNB1 | GPR124 | GPS2 | GREM1 |
| GRIN2A | GRM3 | GSK3B | H3F3A | H3F3B | H3F3C |
| HDAC1 | HGF | HIST1H1C | HIST1H2BD | HIST1H3A | HIST1H3B |
| HIST1H3C | HIST1H3D | HIST1H3E | HIST1H3F | HIST1H3G | HIST1H3H |
| HIST1H3I | HIST1H3J | HIST2H3A | HIST2H3C | HIST2H3D | HIST3H3 |
| HLA-A | HLA-B | HLA-C | HNF1A | HNRNPK | HOXB13 |
| HRAS | HSD3B1 | HSP90AA1 | ICOSLG | ID3 | IDH1 |
| IDH2 | IFNGR1 | IGF1 | IGF1R | IGF2 | IKBKE |
| IKZF1 | IL10 | IL7R | INHA | INHBA | INPP4A |
| INPP4B | INPPL1 | INSR | IRF2 | IRF4 | IRS1 |
| IRS2 | JAK1 | JAK2 | JAK3 | JUN | KAT6A |
| KBTBD4 | KDM5A | KDM5C | KDM6A | KDR | KEAP1 |
| KEL | KIF5B | KIT | KLF4 | KLF5 | KLHL6 |
| KMT2A | KMT2B | KMT2C | KMT2D | KMT5A | KNSTRN |
| KRAS | LAMP1 | LATS1 | LATS2 | LMO1 | LRP1B |
| LTK | LYN | LZTR1 | MAD2L2 | MAGI2 | MALT1 |
| MAP2K1 | MAP2K2 | MAP2K4 | MAP3K1 | MAP3K13 | MAP3K14 |
| MAP3K4 | MAPK1 | MAPK3 | MAPKAP1 | MAX | MCL1 |
| MDC1 | MDM2 | MDM4 | MED12 | MEF2B | MEN1 |
| MET | MGA | MITF | MLH1 | MLLT1 | MLLT3 |
| MPL | MRE11A | MSH2 | MSH3 | MSH6 | MSI1 |
| MSI2 | MST1 | MST1R | MTAP | MTOR | MUTYH |
| MYB | MYC | MYCL | MYCN | MYD88 | MYOD1 |
| NAB2 | NADK | NBN | NCOA3 | NCOR1 | NEGR1 |
| NF1 | NF2 | NFE2L2 | NFKBIA | NKX2-1 | NKX3-1 |
| NOTCH1 | NOTCH2 | NOTCH3 | NOTCH4 | NPM1 | NR-21 |
| NR-27 | NRAS | NRG1 | NSD1 | NT5C2 | NTHL1 |
| NTRK1 | NTRK2 | NTRK3 | NUF2 | NUP93 | NUTM1 |
| P2RY8 | PAK1 | PAK3 | PAK7 | PALB2 | PARK2 |
| PARP1 | PARP2 | PARP3 | PAX3 | PAX5 | PAX7 |
| PAX8 | PBRM1 | PD-1 | PDGFRA | PDGFRB | PDK1 |
| PD-L1 | PD-L2 | PDPK1 | PGBD5 | PGR | PHF6 |
| PHOX2B | PIGA | PIK3C2B | PIK3C2G | PIK3C3 | PIK3CA |
| PIK3CB | PIK3CD | PIK3CG | PIK3R1 | PIK3R2 | PIK3R3 |
| PIM1 | PLCG2 | PLK2 | PMAIP1 | PMS1 | PMS2 |
| PNRC1 | POLD1 | POLE | POT1 | PPARG | PPM1D |
| PPP2R1A | PPP2R2A | PPP4R2 | PPP6C | PRDM1 | PRDM14 |
| PREX2 | PRKAR1A | PRKCI | PRKD1 | PRKDC | PRSS8 |
| PTCH1 | PTEN | PTP4A1 | PTPN11 | PTPRD | PTPRS |
| PTPRT | QKI | RAB35 | RAC1 | RAC2 | RAD21 |
| RAD50 | RAD51 | RAD51B | RAD51C | RAD51D | RAD52 |
| RAD54L | RAF1 | RANBP2 | RARA | RASA1 | RB1 |
| RBM10 | RECQL | RECQL4 | REL | REST | RET |
| RFWD2 | RHEB | RHOA | RICTOR | RIT1 | RNF43 |
| ROS1 | RPS6KA4 | RPS6KB1 | RPS6KB2 | RPTOR | RRAGC |
| RRAS | RRAS2 | RSPO2 | RTEL1 | RUNX1 | RUNX1T1 |
| RXRA | RYBP | SCG5 | SDC4 | SDHA | SDHAF2 |
| SDHB | SDHC | SDHD | SERPINB3 | SERPINB4 | SESN1 |
| SESN2 | SESN3 | SETBP1 | SETD2 | SETDB1 | SF3B1 |
| SGK1 | SH2B3 | SH2D1A | SHOC2 | SHQ1 | SLC34A2 |
| SLFN11 | SLIT2 | SLX4 | SMAD2 | SMAD3 | SMAD4 |
| SMARCA2 | SMARCA4 | SMARCB1 | SMARCD1 | SMARCE1 | SMC1A |
| SMC3 | SMO | SMYD3 | SNCAIP | SOCS1 | SOS1 |
| SOX10 | SOX17 | SOX2 | SOX9 | SPEN | SPOP |
| SPRED1 | SPRTN | SPTA1 | SRC | SRSF2 | STAG1 |
| STAG2 | STAT3 | STAT4 | STAT5A | STAT5B | STK11 |
| STK19 | STK40 | SUFU | SUZ12 | SYK | TAF1 |
| TAP1 | TAP2 | TBX3 | TCEB1 | TCF3 | TCF7L2 |
| TEK | TERT | TET1 | TET2 | TFE3 | TFRC |
| TGFBR1 | TGFBR2 | TIPARP | TMEM127 | TMPRSS2 | TNFAIP3 |
| TNFRSF14 | TOP1 | TOP2A | TP53 | TP53BP1 | TP63 |
| TPMP | TRAF2 | TRAF7 | TRIP13 | TSC1 | TSC2 |
| TSHR | TYRO3 | U2AF1 | UGT1A1 | UPF1 | USP8 |
| VEGFA | VHL | VTCN1 | WHSC1 | WHSC1L1 | WISP3 |
| WT1 | WWTR1 | XIAP | XPO1 | XRCC2 | YAP1 |
| YES1 | ZBTB2 | ZBTB7A | ZFHX3 | ZNF217 | ZNF703 |
| ZNRF3 | ZRSR2 | | | | |

Preferably, said tumour characterisation comprises identifying a genetic aberration relative to said reference genome of at least one coding or non-coding sequence of a gene associated with a cancer treatment, and/or at least one non-coding sequence within a sequence of a gene associated with translocations associated with a cancer treatment and/or at least one splicing region of at least one gene associated with a cancer treatment. This tumour characterisation enables cancer treatment to be better targeted to the specific genetic aberrations of the tumour.

Preferably, said target DNA sequences to be sequenced contain genetic aberrations comprising an allelic imbalance of telomeres relative to said reference genome identified by at least two, preferably at least 100, more preferably at least 1,000, favourably at least 2,000, and preferably at most 20,000, more preferably at most 10,000, favourably at most 6,000 single nucleotide polymorphisms and/or insertions and/or deletions of a nucleotide of at least 1 DNA fragment located in a pre-telomeric region. Advantageously, the single nucleotide polymorphisms are determined with a minor allele frequency equal to or greater than 20%, preferably equal to or greater than 25%, favourably equal to or greater than 30%. An allelic imbalance of telomeres is indicative of a defective DNA repair mechanism and predicts increased sensitivity of the tumour to DNA altering agents such as platinum-based agents. According to the present invention, a pre-telomeric region is defined, for example, by the contents thereof having repetitive DNA sequences and by the fact that it contains no genes.

Preferably, said target DNA sequences to be sequenced contain genetic aberrations comprising a homologous recombination deficiency (HRD) phenotype, said HRD phenotype being determined by (i) identifying a series of single nucleotide polymorphisms of at least one target DNA sequence to be sequenced relative to at least one corresponding DNA sequence of said reference genome, said at least one target DNA sequence to be sequenced being located in at least one genomic region within a gene and, (ii) identifying a series of single nucleotide polymorphisms and/or a series of insertions and/or a series of deletions of a nucleotide by comparing at least one target DNA sequence to be sequenced located in a pre-telomeric region with at least one corresponding DNA sequence located in a corresponding pre-telomeric region of said reference genome, said series of single nucleotide polymorphisms of at least one target DNA sequence to be sequenced located in a pre-telomeric region is determined with a minor allele frequency equal to or greater than 20%, preferably equal to or greater than 25%, favourably equal to or greater than 30%. Analysing the loss of heterozygosity using the combined identification of a single nucleotide polymorphism within a gene and a single nucleotide polymorphism within a pre-telomeric region determined with a sufficient minor allele frequency enables a HRD phenotype to be established while reducing the percentage of false negatives for this phenotype.

Advantageously, said target DNA sequences to be sequenced contain genetic aberrations comprising at least 2, preferably at least 3, more preferably at least 4, even more preferably at least 5, favourably at least 6 genetic aberrations selected from a group of genetic aberrations comprising a single nucleotide polymorphism, a deletion of one or two copies of a gene, a variation in copy number, a homologous recombination deficiency phenotype, a tumour mutational burden, a microsatellite instability, an allelic imbalance of telomeres, or a mutation, translocation or splicing associated with a cancer treatment, said genetic aberrations of the tumour being determined by comparing said target DNA sequences to be sequenced with corresponding DNA sequences of the reference genome. It is advantageous to determine a set of genetic aberrations so as to obtain an accurate and comprehensive tumour characterisation. Identifying a combination of genetic aberrations further enables the sensitivity of the tumour to cancer treatments to be better defined.

Advantageously, said tumour characterisation further comprises identifying an expression of at least one tumour marker measured by its level of RNA and/or microRNA and/or protein. Advantageously, the measurement of the expression of at least one tumour marker at RNA and/or microRNA and/or protein level is a measurement by comparison of the expression of said at least one tumour marker with the expression of said at least one marker in the corresponding healthy tissue from the patient. Tumour characterisation will be all the more comprehensive if, in addition to characterising genetic aberrations, it includes measuring the expression of RNA, microRNA and/or protein markers in the tumour. These expression measurements enhance the genetic characterisation of the tumour to provide a more overall, functional view of the molecular mechanisms occurring in the tumour.

Advantageously, in the characterisation method according to the present invention, said plurality of denatured DNA hybridisation probes specific to said plurality of said blocked DNA fragments is a mixture containing several hybridisation probes of identical or different sequences but complementary to at least 60%, preferably at least 70%, more preferably at least 80%, even more preferably at least 90%, preferably at least 95%, 96%, 97%, 98%, 99% of the sequence of one or more fragments of the plurality said blocked DNA fragments, and wherein said plurality of denatured DNA hybridisation probes specific to said plurality of said blocked DNA fragments is one denatured DNA probe per blocked DNA fragment or several denatured DNA probes per blocked DNA fragment. When the blocked DNA fragment to be enriched is a fragment with a complex sequence, hybridising more than one denatured DNA probe per blocked DNA fragment may improve enrichment efficiency, which increases the enrichment uniformity for both DNA fragments containing simple sequences and DNA fragments containing complex sequences.

Advantageously, said target DNA sequences to be sequenced have an average sequence length between 10 and 10,000 base pairs, preferably between 100 and 1,000 base pairs, more preferably between 200 and 600 base pairs, favourably between 375 and 425 base pairs. Obtaining a set of target DNA sequences to be sequenced with a homogeneous sequence length is a criterion that increases sequencing uniformity.

Advantageously, the characterisation method according to the present invention identifies a treatment based on the tumour characterisation. The treatment identified by the tumour characterisation is specific to the genetic aberrations in the tumour.

Preferably, the characterisation method according to the present invention further comprises tumour mapping wherein the genetic aberrations of the tumour are shown in relation to recommended treatments for the tumours characterised by these genetic aberrations, said recommended treatments being obtained by comparing genetic aberrations in the analysed tumour with genetic aberrations of reference tumours and their reference therapeutic treatments recorded in a database.

Other embodiments of the method according to the present invention are mentioned in the appended claims.

The present invention also relates to an agent or several agents for treating a tumour in a patient whose tumour has been characterised by the method according to the present invention, said agent or agents being selected from the group comprising PARP inhibitors, targeted therapies, immunotherapy, chemotherapy, radiotherapy, DNA-damaging agents such as platinum-based agents, adjuvant therapies, tyrosine kinase inhibitors, immune checkpoint inhibitors, erlotinib, gefitinib, afatinib, osimertinib, dacomitinib, cabozantinib, crizotinib, alectinib, ceritinib, brigatinib, vemurafenib, encorafenib, dabrafenib, trametinib, cobimetinib, docetaxel, paclitaxel, gemcitabine, pemetrexed, pembrolizumab, atezolizumab, nivolumab, durvalumab, olaparib, niraparib, talazoparib, rucaparib, trastuzumab, pertuzumab, neratinib, a treatment or therapy approved or being developed.

Other embodiments of an agent or combination of agents for treating a tumour in a patient according to the present invention are mentioned in the appended claims.

The present invention also relates to the use of a plurality of double-stranded DNA hybridisation probes to carry out targeted sequencing of DNA fragments containing simple sequences and DNA fragments containing complex sequences from a sample from a cancer patient, said sample comprising at least one tumour cell.

Other embodiments of the use of a plurality of double-stranded DNA hybridisation probes according to the present invention are mentioned in the appended claims.

The present invention also relates to a method for treating cancer in a patient comprising the steps of a) characterising the tumour of a patient, b) determining the treatment or treatments to be administered comprising one or more agents selected from the group comprising PARP inhibitors, targeted therapies, immunotherapy, chemotherapy, radiotherapy, DNA-damaging agents such as platinum-based agents, adjuvant therapies, tyrosine kinase inhibitors, immune checkpoint inhibitors, erlotinib, gefitinib, afatinib, osimertinib, dacomitinib, cabozantinib, crizotinib, alectinib, ceritinib, brigatinib, vemurafenib, encorafenib, dabrafenib, trametinib, cobimetinib, docetaxel, paclitaxel, gemcitabine, pemetrexed, pembrolizumab, atezolizumab, nivolumab, durvalumab, olaparib, niraparib, talazoparib, rucaparib, trastuzumab, pertuzumab, neratinib, a treatment or therapy approved or being developed, and c) administering the treatment or treatments to the patient, characterised in that tumour characterisation is implemented by applying the tumour characterisation method according to the present invention.

Advantageously, the method for treating cancer in a patient according to the present invention comprises the step of repeating the tumour characterisation of the patient over time in order to determine whether another treatment should be administered to said patient.

Other embodiments of the method for treating cancer according to the present invention are mentioned in the appended claims.

Lastly, the present invention relates to a theranostic report of a cancer patient, obtained by implementing the tumour characterisation method according to the present invention when it comprises the above-mentioned mapping step, for use in determining a treatment or therapy approved or being developed for the cancer of the patient.

### BRIEF DESCRIPTION OF THE DRAWINGS

Other features, details and advantages of the invention will emerge from the description given below, which is non-limiting and refers to the appended drawings.

FIG. 1 is a schematic representation of the various steps of the method for characterising a tumour by targeted sequencing from a sample from a cancer patient.

FIG. 2 is a diagram of a double-stranded DNA hybridisation probe (double-stranded DNA probe).

FIG. 3 is a diagram explaining the use of a plurality of double-stranded DNA hybridisation probes in oncology to carry out targeted sequencing of a tumour.

FIG. 4 shows a diagram showing the steps for using the tumour characterisation method in order to identify a suitable treatment and establish a theranostic report of the tumour.

In the figures, the same or like items bear the same references. Black arrows indicate the direction of progress to be followed.

### DETAILED DESCRIPTION OF THE INVENTION

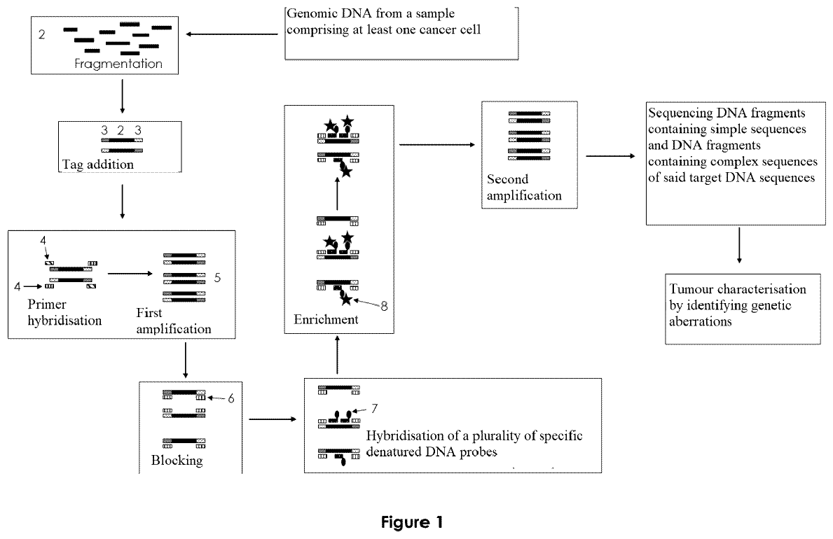

FIG. 1 shows an example of the various steps of the method for characterising a tumour by targeted sequencing from a sample from a cancer patient. Each box in FIG. 1 shows a step in a succession of steps of the tumour characterisation method. In a first step 1, the genomic DNA from a sample comprising at least one cancer cell, genomic DNA of the tumour, is harvested. Preferably, the sample may be any solid tumour sample such as formalin-fixed paraffin-embedded (FFPE) tissue or frozen tissue. Preferably, the initial amount of genomic DNA used for the method is equal to or greater than 10 ng, more preferably approximately 50 ng. This genomic DNA is then fragmented, in a fragmentation step, to give DNA fragments 2. This fragmentation step may be carried out by physical or mechanical fragmentation, such as sonication, or enzymatic fragmentation, such as the use of endonuclease, or chemical fragmentation. The duration of the fragmentation step may be between 1 min and 1 h, preferably between 5 mins and 25 mins. Tags are then added to these DNA fragments 2. For this, these DNA fragments 2 undergo a sequence end repair step so that there are no unpaired nucleotides (step not shown in FIG. 1). These DNA fragments 2 are then polyadenylated (step not shown in FIG. 1) and will undergo a universal adapter ligation step. The DNA fragment end repair, polyadenylation and universal adapter ligation steps are schematised overall by the tag addition step 3 shown in FIG. 1. The tags comprising universal adapters enable primers to be hybridised, among other things. These DNA fragments with the added tags 3, may then be selected according to their size (step not shown in FIG. 1). Preferably, the DNA fragments to be sequenced have an average sequence length between 10 and 10,000 base pairs, preferably between 100 and 1,000 base pairs, more preferably between 200 and 600 base pairs, favourably between 375 and 425 base pairs. DNA fragments with a similar sequence length make targeted sequencing more uniform. The harvested DNA fragments are then amplified in a first amplification step 5. This first amplification step may be a PCR amplification. For this, PCR primers 4 having a sequence complementary to at least one portion of a sequence of tags will hybridise to the tags and initiate the first amplification 5. The fragmentation 2, tag addition 3, PCR primer hybridisation 4 and first amplification 5 steps enable a sequencing library to be prepared and give a first mixture comprising amplified DNA fragments.

The DNA fragments amplified by the first amplification 5 are then blocked, in a blocking step, using universal blockers 6. The amplified DNA fragments are blocked using universal blockers 6 having a sequence complementary to at least one portion of the sequence of tags. These universal blockers 6 prevent non-specific hybridisation between the sequences of tags and therefore enable better subsequent enrichment of the target DNA fragments. Furthermore, the genome comprises a lot of repetitive DNA sequences that should be removed before sequencing. In the blocking step, adding Cot-1 DNA in addition to universal blockers 6 enables repetitive DNA sequences in the genome to be removed (step not shown in FIG. 1). Cot-1 DNA is a sample containing DNA sequences complementary to most of the repetitive DNA sequences in the human genome and practically none of the unique (non-repetitive) sequences in the human genome. Cot-1 DNA therefore enables a large portion of repetitive sequences in the genome to be removed by complementarity hybridisation. The average length of DNA fragments in such a sample is approximately 300 base pairs (bp). Cot-1 DNA is used to remove repetitive DNA sequences in genomic hybridisation.

Next, FIG. 1 shows the hybridisation of a plurality of said DNA fragments blocked by said plurality of denatured DNA hybridisation probes specific to said plurality of said blocked DNA fragments to form a second mixture comprising DNA fragments hybridised and DNA fragments not hybridised to the plurality of double-stranded DNA hybridisation probes (point 7 in FIG. 1). The plurality of specific denatured DNA hybridisation probes derived from the denaturation of the plurality of specific double-stranded DNA hybridisation probes.

Prior to the hybridisation step between said blocked DNA fragments and said plurality of specific denatured DNA hybridisation probes, the double-stranded DNA hybridisation probes are denatured, preferably by heating to a temperature advantageously between 95° C. and 99° C., to form denatured DNA hybridisation probes. In the context of the present invention, hybridisation between a double-stranded DNA hybridisation probe and a DNA fragment from the sample of the patient means hybridisation between a double-stranded DNA probe that has been previously denatured into two single-stranded DNA probes wherein each single-stranded DNA probe previously forming the double-stranded DNA probe hybridises to a strand of the DNA fragment, target DNA sequence, from the sample from the patient. In a particular embodiment of the method according to the present invention, this step of hybridising a plurality of said DNA fragments blocked by a plurality of denatured DNA hybridisation probes specific to said plurality of said blocked DNA fragments is carried out at a temperature between 40 and 90° C., preferably between 60 and 80° C., favourably at 70° C., for a time period between 1 h and 40 h, preferably between 10 and 20 h, favourably 16 h.

An enrichment step (point 8 in FIG. 1) is carried out by uniformly and simultaneously enriching DNA fragments containing simple sequences and DNA fragments containing complex sequences of said DNA fragments hybridised to the plurality of denatured DNA hybridisation probes of said second mixture by capturing said hybridised DNA fragments to form a medium enriched with said DNA fragments hybridised to the plurality of denatured DNA hybridisation probes. Favourably, the complex sequences are defined by a percentage of guanine and cytosine nucleotide base equal to or greater than 80%. Favourably, the blocking step (point 6 in FIG. 1) and the hybridisation step of blocked DNA fragments carried out using a plurality of specific denatured DNA hybridisation probes (point 7 in FIG. 1) are carried out concomitantly. Advantageously, hybridised DNA fragments are captured using magnetic streptavidin beads (point 8 in FIG. 1). These streptavidin beads are superparamagnetic particles covalently coupled to streptavidin proteins. Hybridised DNA fragments are captured by applying a magnetic field and at a very high affinity existing between a streptavidin protein and a biotin molecule, which enables only DNA fragments hybridised to the denatured DNA hybridisation probes to be retained. Preferably, the ratio between the amount of streptavidin beads and the amount of double-stranded DNA hybridisation probes is between 0.5 and 3, more preferably between 1 and 2, favourably 1.4. The incubation time for DNA fragments hybridised to the pairings of denatured DNA probes-biotin with streptavidin beads is between 1 min and 5 h, preferably between 10 min and 1 h, favourably 30 min.

After the enrichment step, a step of washing and recovering said DNA fragments hybridised to the plurality of denatured DNA hybridisation probes enables a medium to be formed, enriched with target DNA sequences. This step of washing and recovering is carried out between the steps of enriching a sample of DNA to be sequenced and the second amplification. In particular, one or more washing steps followed by a recovery step may be carried out (steps not shown in FIG. 1). These steps ensure optimal purification of DNA fragments to be sequenced. Preferably, a first washing step is carried out with a first washing buffer having a temperature between 20 and 30° C., favourably 25° C., followed by a second washing step with a second washing buffer having a temperature between 40 and 60° C., favourably 48° C.

After the washing and recovery steps, which remove blockers and anything that is not hybridised to the plurality of denatured DNA hybridisation probes (steps not shown in FIG. 1), a second amplification, post-capture PCR amplification, is carried out (see FIG. 1). This second amplification of said target DNA sequences is carried out using primers having a sequence complementary to a sequence of said tags giving target DNA sequences to be sequenced. This second amplification makes it possible to obtain sufficient target DNA sequences to be sequenced for subsequent sequencing. Preferably, this second amplification involves between 5 and 15 PCR cycles, favourably 9 PCR cycles.

Favourably, the various steps described above make it possible to obtain a sample comprising target DNA sequences to be sequenced of a high quality defined by a concentration of target DNA sequences to be sequenced equal to or greater than 15 ng/μl and by target DNA sequences to be sequenced having a sequence defined by an average length between 375 and 425 base pairs.

After the second amplification, a targeted sequencing of target DNA sequences to be sequenced is carried out (see FIG. 1). This targeted sequencing, which generates sequencing data, is a simultaneous sequencing of DNA fragments containing simple sequences and DNA fragments containing complex sequences under the same sequencing conditions. Advantageously, the sequencing is carried out by a NextSeq500/550 sequencer or any Illumina or MGI sequencer. Preferably, the sequencing is a paired end sequencing, but may also be a single end sequencing.

Using bioinformatics analysis algorithms, such as
ABRA (https://academic.oup.com/bioinformatics/article/30/19/2813/2422200),
SAMTOOLS (https://www.ncbi.nlm.nih.gov/pmc/articles/PMC2723002/),
BEDTOOLS (https://academic.oup.com/bioinformatics/article/26/6/841/244688), or
FASTP (https://academic.oup.com/bioinformatics/article/34/17/1884/5093234),
   the data obtained by targeted sequencing enabled the tumour to be characterised by sequencing DNA fragments containing simple sequences and DNA fragments containing complex sequences of said target DNA sequences to be sequenced, so as to identify genetic aberrations in the tumour (see the last step in FIG. 1).

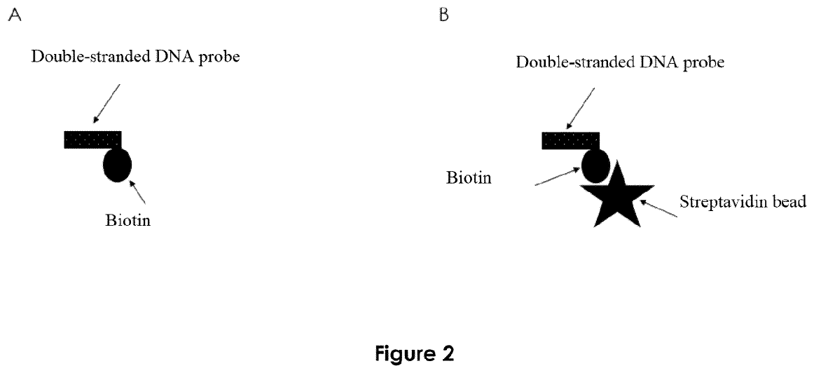

FIG. 2 shows a schematic representation of a double-stranded DNA hybridisation probe (double-stranded DNA probe) coupled to a biotin molecule (point A in FIG. 2). Point B in FIG. 2 shows the affinity between the biotin molecule and the streptavidin protein which is covalently linked to a superparamagnetic particle to form the streptavidin bead. The DNA fragment hybridised to the double-stranded DNA hybridisation probe which will be enriched in order to be sequenced is not shown in FIG. 2.

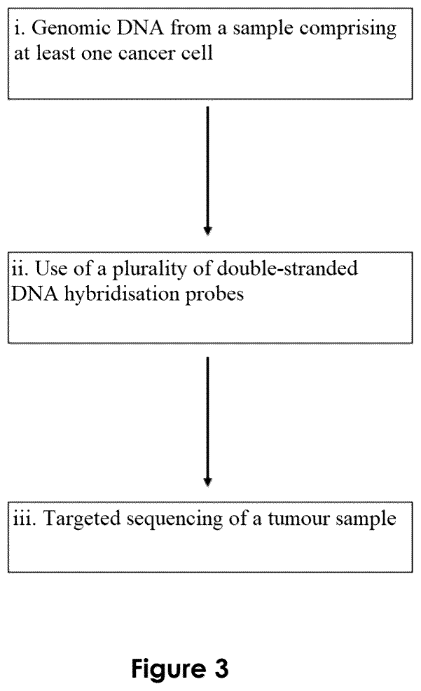

FIG. 3 shows the use of a plurality of double-stranded DNA hybridisation probes to carry out targeted sequencing of DNA fragments containing simple sequences and DNA fragments containing complex sequences from a sample from a cancer patient, said sample comprising at least one tumour cell. Each box in FIG. 3 shows a step for using a plurality of double-stranded DNA hybridisation probes in oncology. Using a plurality of double-stranded DNA hybridisation probes ii derived from genomic DNA from a sample comprising at least one cancer cell i enables targeted sequencing of the tumour iii to be carried out. In particular, using a plurality of double-stranded DNA hybridisation probes on a DNA sample derived from a tumour enables uniform enrichment and targeted sequencing of the tumour, which limits sequencing costs while decreasing the number of false negatives and false positives in the sequencing data.

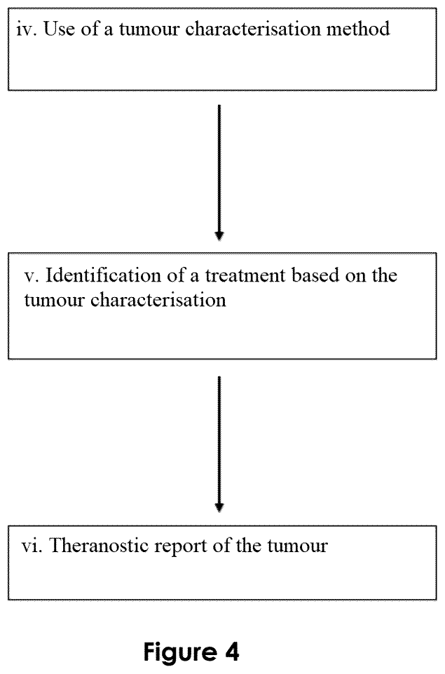

FIG. 4 shows the steps for using the method described above for characterising a tumour iv to identify a treatment based on the characterisation of the tumour v. Furthermore, by using the tumour characterisation method described above, which enables genetic aberrations in the tumour to be identified and a suitable treatment based on the tumour characterisation to be predicted, a theranostic report of the tumour may be generated vi. Each box in FIG. 4 shows a step in using the characterisation method to identify a suitable treatment and to establish a theranostic report of the tumour.

Advantageously, the theranostic report of the tumour may comprise medical information and/or targeted sequencing data of the tumour with a list of the genetic aberrations identified and/or additional tumour characterisation data such as measurements of tumour marker expression at RNA and/or microRNA and/or protein level, and/or an immunogram defining a potential to respond to immunotherapy based on the tumour characterisation, and/or a list of treatments with their properties which may be associated with a clinical benefit and/or those which would not be associated with a benefit, and/or a list of clinical trials associated with the tumour characterisation and/or a list of publications related to the tumour characterisation.

### EXAMPLES

### Example 1

### Harvesting Genomic DNA From a Sample From a Solid Tumour and Preparing This DNA for Sequencing

In a first step in the method for characterising a tumour according to the present invention, genomic DNA was harvested from a sample comprising at least one cancer cell. For this, the following steps were implemented:
receiving a sample composed of a block comprising a formalin-fixed paraffin-embedded (FFPE block) piece of tumour extracted by biopsy;
cutting the block into slides with a thickness of 7 μm;
staining the first and last slide with hematoxylin and eosin (H&E slides);
identifying from the first slide, by visual inspection, an area of the slide comprising sufficient tumour cells, little or no necrosis and lymphocytic infiltration of less than 21%;
marking a region comprising the most tumour cells on this stained slide,
transferring the marking from the first slide to the unstained slides to mark the same tumour region, but on a different slice;
scraping the cells in these marked areas (macrodissection step);
extracting DNA from these slides to form the genomic DNA to be characterised;
in parallel, quantifying, using a spectrophotometer, DNA from a sample of genomic DNA to be characterised;
in parallel, qualifying DNA by gel migration from a sample of genomic DNA to be characterised;

The genomic DNA sample may then be characterised by applying the protocol in FIG. 1.

### Example 2

### Enriching DNA Fragments Containing Simple Sequences and DNA Fragments Containing Complex Sequences of Said DNA Fragments Hybridised to the Plurality of Denatured DNA Hybridisation Probes

To enrich DNA fragments containing simple sequences and DNA fragments containing complex sequences of said DNA fragments hybridised to the plurality of denatured DNA hybridisation probes, the protocol in FIG. 1 was applied to the tumour sample in Example 1. More precisely, 50 ng of genomic DNA from the sample was fragmented by sonication for 10 minutes, which gave DNA fragments. Next, tags were added to the ends of DNA fragments using a standard protocol well known to those skilled in the art. These DNA fragments were then amplified in a first PCR amplification step comprising 8 PCR amplification cycles to give amplified DNA fragments. These amplified DNA fragments were then simultaneously blocked using universal blockers and hybridised using double-stranded DNA hybridisation probes where the hybridisation time was 16 h at 70° C. to give hybridised DNA fragments. Prior to the hybridisation step, a step of denaturing by heating to a temperature of 98° C. for 15 seconds was carried out to denature the DNA fragments and the double-stranded DNA hybridisation probes, forming a mixture of single-stranded DNA fragments and single-stranded denatured DNA hybridisation probes.

Subsequently, the hybridised DNA fragments were captured using magnetic streptavidin beads where the incubation time with the beads was 30 minutes with a bead ratio relative to the double-stranded DNA hybridisation probes of 1.4 to give a mixture enriched with DNA fragments hybridised to the plurality of denatured DNA hybridisation probes. The DNA fragments hybridised to the plurality of denatured DNA hybridisation probes were then washed using two washing steps and recovered to form a medium enriched with target DNA sequences. The first washing step was carried out at a temperature of 25° C. whereas the second washing step was carried out at a temperature of 48° C. Next, the target DNA sequences were amplified by a second PCR amplification using 9 PCR amplification cycles to give a sample enriched with target DNA sequences to be sequenced. Lastly, a quality control of the sample enriched with target DNA sequences to be sequenced was carried out, where the concentration of DNA from the sample enriched with target DNA sequences to be sequenced was 16 ng/μl with an average size of target DNA sequences to be sequenced of 380 bp.

By carrying out these various steps in Example 2, a uniform enrichment, defined by a uniformity greater than 90%, of DNA fragments containing simple sequences and DNA fragments containing complex sequences of said DNA fragments hybridised to the plurality of denatured DNA hybridisation probes could be obtained and wherein a percentage of duplicated sequences was less than 16%.

### Example 3

### Targeted Sequencing of Target DNA Sequences to be Sequenced From a solid tumour sample.

Starting from the sample enriched with the obtained target DNA sequences to be sequenced (see Example 2), a sequencing was carried out using a NextSeq500 sequencer wherein the flow cell loading concentration was 1.6 pM and wherein the sequencing carried out was paired end sequencing: 2*75 bp. The average sequencing coverage was at least 400. This sequencing yielded a cluster density of approximately 265,000/mm² with a cluster passing filter of 84.5% for a total sequencing read count of approximately 228,000,000 (228 million).

### Example 4

### Characterising Genetic Aberrations in a Solid Tumour Sample

Using bioinformatic analysis, the targeted sequencing data obtained (see Example 3) were compared with a reference sequence (hg19, grch37) from NCBI: https://www.ncbi.nlm.nih.gov/search/all/?term=grch37.p13.), which enabled genetic aberrations in the tumour to be identified. Next, a theranostic report was carried out comprising medical information, targeted sequencing data of the tumour with a list of the genetic aberrations identified, an immunogram defining a potential to respond to immunotherapy based on the tumour characterisation, a list of treatments with their properties which may be associated with a clinical benefit and those that would not be associated with a benefit, a list of clinical trials associated with the tumour characterisation and a list of publications related to the tumour characterisation.

The total analysis time comprising harvesting genomic DNA and preparing this genomic DNA (Example 1), enriching DNA fragments containing simple sequences and DNA fragments containing complex sequences (Example 2), targeted sequencing of target DNA sequences to be sequenced (Example 3) and charactering the tumour by identifiying genetic aberrations in the tumour and establishing a theranostic report of the tumour (Example 4 above) was 10 working days.

### Comparative Example 1

### Comparison of Exome Sequencing

The exemplified exome sequencing compares 2 methods:
1. one method according to the present invention, using double-stranded DNA hybridisation probes for exomes for the hybridisation and enrichment steps (see the protocol of FIG. 1 and Example 2), and
2. one method according to Agilent technology that uses next-generation sequencing based on target enrichment by hybridisation capture using probes for exomes (SureSelect Focused Exome and SureSelect Human All Exome).

TABLE 2
Specificity characteristics of sequencing according
to the method according to the present invention
and according to Agilent technology.

| | Sequencing | |
|---|---|---|
| | according to the | Agilent |
| | present invention | technology |
| Baits (sequences) covered | 36695332 | 65962670 |
| (sequencing coverage) | | |
| Size of the targeted sequences | 33359393 | 45659296 |
| Percentage of baits | 90% | 70% |
| among target sequences | | |

Table 2 shows the specificity characteristics of sequencing according to the 2 methods described above. The proportion of the size of the targeted sequences relative to the sequencing coverage is greater (90% compared with 70%) in the method according to the present invention using double-stranded DNA hybridisation probes, which demonstrates greater sequencing specificity using the method according to the present invention. This greater specificity makes it possible to minimise the sequencing depth to obtain a given amount of sequencing information, thus accelerating sequencing and reducing sequencing costs.

Regarding sequencing uniformity, the method according to the present invention, using double-stranded DNA hybridisation probes for exomes, enables an average sequencing depth of 70 times, where 99% of targets were covered more than 25 times, 79% of targets were covered more than 50 times and only 3% of targets were covered more than 100 times. By comparison, with Agilent technology, the average sequencing depth is 64 times where 93% of targets were covered more than 25 times, 66% of targets were covered more than 50 times and 12% of targets were covered more than 100 times. The uniformity of the sequencing coverage is therefore greater when using double-stranded DNA hybridisation probes according to the present invention.

It is to be understood that the present invention is in no way limited to the embodiments described above and that modifications may be made without departing from the scope of the appended claims.

1. A method for characterizing a tumor by targeted sequencing from a sample from a cancer patient, comprising the steps of:
harvesting genomic DNA from said sample comprising at least one cancer cell,
preparing a sequencing library from said sample comprising the successive steps of (i) fragmenting said genomic DNA, generating DNA fragments, (ii) adding a series of tags to ends of said DNA fragments, (iii) a first amplification of said DNA fragments using primers having a sequence complementary to a sequence of said tags forming a first mixture comprising amplified DNA fragments,
blocking said amplified DNA fragments of said first mixture using a series of blockers having a sequence complementary to at least one portion of the sequence of tags to form blocked DNA fragments, and
sequencing and characterizing the tumor,
wherein said sequencing and said tumor characterization comprise the steps of:
collecting a plurality of double-stranded DNA hybridization probes specific to a plurality of said blocked DNA fragments,
denaturing said double-stranded DNA hybridization probes to form a plurality of denatured DNA hybridization probes specific to said plurality of said blocked DNA fragments,
hybridizing said plurality of said blocked DNA fragments by said plurality of denatured DNA hybridization probes to form a second mixture comprising DNA fragments hybridized and DNA fragments not hybridized to said plurality of denatured DNA hybridization probes,
uniformly and simultaneously enriching the second mixture with DNA fragments containing simple sequences and with DNA fragments containing complex sequences of said DNA fragments hybridized to the plurality of denatured DNA hybridization probes by capturing said hybridized DNA fragments to form a medium enriched with said DNA fragments hybridized to the plurality of denatured DNA hybridization probes,
washing and recovering said DNA fragments hybridized to the plurality of denatured DNA hybridization probes to form a medium enriched with target DNA sequences,
a second amplification of said target DNA sequences using primers having a sequence complementary to a sequence of said tags providing target DNA sequences to be sequenced,
sequencing DNA fragments containing simple sequences and DNA fragments containing complex sequences of said target DNA sequences to be sequenced so as to identify genetic aberrations in the tumor.

2. The method according to claim 1, wherein said complex sequences of said target DNA sequences to be sequenced are sequences with a percentage of guanine and cytosine nucleotide base equal to or greater than 60%, or repetitive sequences or inverted sequences.

3. The method according to claim 1, wherein said DNA fragments hybridized to said plurality of denatured DNA hybridization probes have a sequence of which at least a portion is a coding sequence of said DNA fragments containing simple sequences or said DNA fragments containing complex sequences and/or a sequence of which at least a portion is a non-coding sequence of said DNA fragments containing simple sequences or said DNA fragments containing complex sequences.

4. The method according to claim 1, wherein said target DNA sequences to be sequenced contain genetic aberrations comprising a series of single nucleotide polymorphisms of one or more DNA sequences relative to one or more corresponding DNA sequences of a reference genome, said series of single nucleotide polymorphisms preferably comprising at least 50 single nucleotide polymorphisms, said single nucleotide polymorphisms being located, on average over the whole genome, every 0.5 to 50 megabases.

5. The method according to claim 1, wherein said target DNA sequences to be sequenced contain genetic aberrations comprising a deletion of one or two copies of a gene relative to said reference genome.

6. The method according to claim 1, wherein said target DNA sequences to be sequenced contain genetic aberrations comprising a variation in copy number relative to a copy number of said reference genome associated with at least 2 genomic regions randomly distributed in the genome.

7. The method according to claim 1, wherein said target DNA sequences to be sequenced contain genetic aberrations comprising a homologous recombination deficiency phenotype determined by comparing at least one of said target DNA sequences to be sequenced with at least one corresponding DNA sequence of said reference genome.

8. The method according to claim 1, wherein said target DNA sequences to be sequenced contain genetic aberrations comprising a tumor mutational burden determined by comparing a plurality of coding sequences of said target DNA sequences to be sequenced with a plurality of corresponding coding sequences of said reference genome, said plurality of coding sequences of said target DNA sequences to be sequenced being determined by sequencing at least one megabase of coding sequences of said target DNA sequences to be sequenced.

9. The method according to claim 1, wherein said target DNA sequences to be sequenced contain genetic aberrations comprising a microsatellite instability determined by comparing a series of said target DNA sequences to be sequenced with a series of corresponding DNA sequences of said reference genome, said series of said target DNA sequences to be sequenced is determined by sequencing a least one target DNA sequence to be sequenced.

10. The method according to claim 1, wherein said tumor characterization comprises sequencing at least one portion of a sequence of at least 100 of each gene of a panel of genes consisting of:

| | | | | | |
|---|---|---|---|---|---|
| ABL1 | ABL2 | ACVR1 | ACVR1B | AGO1 | AGO2 |
| AJUBA | AKT1 | AKT2 | AKT3 | ALB | ALK |
| ALOX12B | AMER1 | ANKRD11 | ANKRD26 | APC | APLNR |
| AR | ARAF | ARFRP1 | ARHGAP35 | ARID1A | ARID1B |
| ARID2 | ARID5B | ASXL1 | ASXL2 | ATM | ATR |
| ATRX | ATXN7 | AURKA | AURKB | AXIN1 | AXIN2 |
| AXL | B2M | BABAM1 | BAP1 | BARD1 | BAT25 |
| BAT-26 | BBC3 | BCL10 | BCL2 | BCL2L1 | BCL2L11 |
| BCL2L2 | BCL6 | BCOR | BCORL1 | BCR | BIRC3 |
| BLM | BMPR1A | BRAF | BRCA1 | BRCA2 | BRD4 |
| BRIP1 | BTG1 | BTG2 | BTK | C11orf30 | CALR |
| CARD11 | CARM1 | CASP8 | CBFB | CBL | CCNB3 |
| CCND1 | CCND2 | CCND3 | CCNE1 | CD276 | CD70 |
| CD74 | CD79A | CD79B | CDC42 | CDC73 | CDH1 |
| CDK12 | CDK4 | CDK6 | CDK7 | CDK8 | CDKN1A |
| CDKN1B | CDKN2A | CDKN2B | CDKN2C | CEBPA | CENPA |
| CHD2 | CHD4 | CHEK1 | CHEK2 | CIC | CMTR2 |
| CREBBP | CRKL | CRLF2 | CSDE1 | CSF1R | CSF3R |
| CSNK1A1 | CTCF | CTLA4 | CTNNA1 | CTNNB1 | CTR9 |
| CUL3 | CUL4A | CUX1 | CXCR4 | CYLD | CYP17A1 |
| CYP19A1 | CYP2C19 | CYP2D6 | CYSLTR2 | D2S123 | DAXX |
| DCUN1D1 | DDR1 | DDR2 | DDX41 | DHX15 | DICER1 |
| DIS3 | DNAJB1 | DNMT1 | DNMT3A | DNMT3B | DOT1L |
| DPYD | DROSHA | DUSP4 | E2F3 | EED | EGFL7 |
| EGFR | EIF1AX | EIF4A2 | EIF4E | ELF3 | EML4 |
| EMSY | EP300 | EPAS1 | EPCAM | EPHA3 | EPHA5 |
| EPHA7 | EPHB1 | EPHB4 | ERBB2 | ERBB3 | ERBB4 |
| ERCC1 | ERCC2 | ERCC3 | ERCC4 | ERCC5 | ERF |
| ERG | ERRFI1 | ESR1 | ETAA1 | ETS1 | ETV1 |
| ETV4 | ETV5 | ETV6 | EWSR1 | EZH1 | EZH2 |
| EZR | FAM175A | FAM46C | FAM58A | FANCA | FANCC |
| FANCD2 | FANCE | FANCF | FANCG | FANCI | FANCL |
| FAS | FAT1 | FBXW7 | FGF1 | FGF10 | FGF12 |
| FGF14 | FGF19 | FGF2 | FGF23 | FGF3 | FGF4 |
| FGF5 | FGF6 | FGF7 | FGF8 | FGF9 | FGFR1 |
| FGFR2 | FGFR3 | FGFR4 | FH | FLCN | FLI1 |
| FLT1 | FLT3 | FLT4 | FOXA1 | FOXF1 | FOXL2 |
| FOXO1 | FOXP1 | FRS2 | FUBP1 | FYN | GAB1 |
| GAB2 | GABRA6 | GATA1 | GATA2 | GATA3 | GATA4 |
| GATA6 | GEN1 | GID4 | GLI1 | GNA11 | GNA13 |
| GNAQ | GNAS | GNB1 | GPR124 | GPS2 | GREM1 |
| GRIN2A | GRM3 | GSK3B | H3F3A | H3F3B | H3F3C |
| HDAC1 | HGF | HIST1H1C | HIST1H2BD | HIST1H3A | HIST1H3B |
| HIST1H3C | HIST1H3D | HIST1H3E | HIST1H3F | HIST1H3G | HIST1H3H |
| HIST1H3I | HIST1H3J | HIST2H3A | HIST2H3C | HIST2H3D | HIST3H3 |
| HLA-A | HLA-B | HLA-C | HNF1A | HNRNPK | HOXB13 |
| HRAS | HSD3B1 | HSP90AA1 | ICOSLG | ID3 | IDH1 |
| IDH2 | IFNGR1 | IGF1 | IGF1R | IGF2 | IKBKE |
| IKZF1 | IL10 | IL7R | INHA | INHBA | INPP4A |
| INPP4B | INPPL1 | INSR | IRF2 | IRF4 | IRS1 |
| IRS2 | JAK1 | JAK2 | JAK3 | JUN | KAT6A |
| KBTBD4 | KDM5A | KDM5C | KDM6A | KDR | KEAP1 |
| KEL | KIF5B | KIT | KLF4 | KLF5 | KLHL6 |
| KMT2A | KMT2B | KMT2C | KMT2D | KMT5A | KNSTRN |
| KRAS | LAMP1 | LATS1 | LATS2 | LMO1 | LRP1B |
| LTK | LYN | LZTR1 | MAD2L2 | MAGI2 | MALT1 |
| MAP2K1 | MAP2K2 | MAP2K4 | MAP3K1 | MAP3K13 | MAP3K14 |
| MAP3K4 | MAPK1 | MAPK3 | MAPKAP1 | MAX | MCL1 |
| MDC1 | MDM2 | MDM4 | MED12 | MEF2B | MEN1 |
| MET | MGA | MITF | MLH1 | MLLT1 | MLLT3 |
| MPL | MRE11A | MSH2 | MSH3 | MSH6 | MSI1 |
| MSI2 | MST1 | MST1R | MTAP | MTOR | MUTYH |
| MYB | MYC | MYCL | MYCN | MYD88 | MYOD1 |
| NAB2 | NADK | NBN | NCOA3 | NCOR1 | NEGR1 |
| NF1 | NF2 | NFE2L2 | NFKBIA | NKX2-1 | NKX3-1 |
| NOTCH1 | NOTCH2 | NOTCH3 | NOTCH4 | NPM1 | NR-21 |
| NR-27 | NRAS | NRG1 | NSD1 | NT5C2 | NTHL1 |
| NTRK1 | NTRK2 | NTRK3 | NUF2 | NUP93 | NUTM1 |
| P2RY8 | PAK1 | PAK3 | PAK7 | PALB2 | PARK2 |
| PARP1 | PARP2 | PARP3 | PAX3 | PAX5 | PAX7 |
| PAX8 | PBRM1 | PD-1 | PDGFRA | PDGFRB | PDK1 |
| PD-L1 | PD-L2 | PDPK1 | PGBD5 | PGR | PHF6 |
| PHOX2B | PIGA | PIK3C2B | PIK3C2G | PIK3C3 | PIK3CA |
| PIK3CB | PIK3CD | PIK3CG | PIK3R1 | PIK3R2 | PIK3R3 |
| PIM1 | PLCG2 | PLK2 | PMAIP1 | PMS1 | PMS2 |
| PNRC1 | POLD1 | POLE | POT1 | PPARG | PPM1D |
| PPP2R1A | PPP2R2A | PPP4R2 | PPP6C | PRDM1 | PRDM14 |
| PREX2 | PRKAR1A | PRKCI | PRKD1 | PRKDC | PRSS8 |
| PTCH1 | PTEN | PTP4A1 | PTPN11 | PTPRD | PTPRS |
| PTPRT | QKI | RAB35 | RAC1 | RAC2 | RAD21 |
| RAD50 | RAD51 | RAD51B | RAD51C | RAD51D | RAD52 |
| RAD54L | RAF1 | RANBP2 | RARA | RASA1 | RB1 |
| RBM10 | RECQL | RECQL4 | REL | REST | RET |
| RFWD2 | RHEB | RHOA | RICTOR | RIT1 | RNF43 |
| ROS1 | RPS6KA4 | RPS6KB1 | RPS6KB2 | RPTOR | RRAGC |
| RRAS | RRAS2 | RSPO2 | RTEL1 | RUNX1 | RUNX1T1 |
| RXRA | RYBP | SCG5 | SDC4 | SDHA | SDHAF2 |
| SDHB | SDHC | SDHD | SERPINB3 | SERPINB4 | SESN1 |
| SESN2 | SESN3 | SETBP1 | SETD2 | SETDB1 | SF3B1 |
| SGK1 | SH2B3 | SH2D1A | SHOC2 | SHQ1 | SLC34A2 |
| SLFN11 | SLIT2 | SLX4 | SMAD2 | SMAD3 | SMAD4 |
| SMARCA2 | SMARCA4 | SMARCB1 | SMARCD1 | SMARCE1 | SMC1A |
| SMC3 | SMO | SMYD3 | SNCAIP | SOCS1 | SOS1 |
| SOX10 | SOX17 | SOX2 | SOX9 | SPEN | SPOP |
| SPRED1 | SPRTN | SPTA1 | SRC | SRSF2 | STAG1 |
| STAG2 | STAT3 | STAT4 | STAT5A | STAT5B | STK11 |
| STK19 | STK40 | SUFU | SUZ12 | SYK | TAF1 |
| TAP1 | TAP2 | TBX3 | TCEB1 | TCF3 | TCF7L2 |
| TEK | TERT | TET1 | TET2 | TFE3 | TFRC |
| TGFBR1 | TGFBR2 | TIPARP | TMEM127 | TMPRSS2 | TNFAIP3 |
| TNFRSF14 | TOP1 | TOP2A | TP53 | TP53BP1 | TP63 |
| TPMP | TRAF2 | TRAF7 | TRIP13 | TSC1 | TSC2 |
| TSHR | TYRO3 | U2AF1 | UGT1A1 | UPF1 | USP8 |
| VEGFA | VHL | VTCN1 | WHSC1 | WHSC1L1 | WISP3 |
| WT1 | WWTR1 | XIAP | XPO1 | XRCC2 | YAP1 |
| YES1 | ZBTB2 | ZBTB7A | ZFHX3 | ZNF217 | ZNF703 |
| ZNRF3 | ZRSR2 | | | | |

or sequencing at least one portion of a sequence of each gene of a subgroup of genes included in the panel of genes to identify a signature of genetic aberrations associated with a therapy.

11. The method according to claim 1, wherein said tumor characterization comprises identifying a genetic aberration relative to said reference genome of at least one coding or non-coding sequence of a gene associated with a cancer treatment, and/or at least one non-coding sequence within a sequence of a gene associated with translocations associated with a cancer treatment and/or at least one splicing region of at least one gene associated with a cancer treatment.

12. The method according to claim 1, wherein said target DNA sequences to be sequenced contain genetic aberrations comprising an allelic imbalance of telomeres relative to said reference genome identified by at least two single nucleotide polymorphisms and/or insertions and/or deletions of a nucleotide of at least 1 DNA fragment located in a pre-telomeric region.

13. The method according to claim 12, wherein said single nucleotide polymorphisms of at least 1 DNA fragment located in a pre-telomeric region are determined with a minor allele frequency equal to or greater than 20%.

14. The method according to claim 1, wherein said target DNA sequences to be sequenced contain genetic aberrations comprising a homologous recombination deficiency (HRD) phenotype, said HRD phenotype being determined by (i) identifying a series of single nucleotide polymorphisms of at least one target DNA sequence to be sequenced relative to at least one corresponding DNA sequence of said reference genome, said at least one target DNA sequence to be sequenced being located in at least one genomic region within a gene and, (ii) identifying a series of single nucleotide polymorphisms and/or a series of insertions and/or a series of deletions of a nucleotide by comparing at least one target DNA sequence to be sequenced located in a pre-telomeric region with a least one corresponding DNA sequence located in a corresponding pre-telomeric region of said reference genome, said series of single nucleotide polymorphisms of at least one target DNA sequence to be sequenced located in a pre-telomeric region is determined with a minor allele frequency equal to or greater than 20%.

15. The method according to claim 1, wherein said target DNA sequences to be sequenced contain genetic aberrations comprising at least 2, genetic aberrations selected from a group of genetic aberrations comprising a single nucleotide polymorphism, a deletion of one or two copies of a gene, a variation in copy number, a homologous recombination deficiency phenotype, a mutational burden, a microsatellite instability, an allelic imbalance of telomeres, or a mutation, translocation or splicing associated with a cancer treatment, said genetic aberrations being determined by comparing said target DNA sequences to be sequenced with corresponding DNA sequences of the reference genome.

16. The method according to claim 1, wherein said tumor characterization further comprises identifying an expression of at least one tumor marker measured by its level of RNA and/or microRNA and/or protein.

17. The method according to claim 1, wherein said plurality of denatured DNA hybridization probes specific to said plurality of said blocked DNA fragments is a mixture containing a plurality of hybridization probes of identical or different sequences but complementary to at least 60% of the sequence of one or more fragments of the plurality of said blocked DNA fragments, and wherein said plurality of denatured DNA hybridization probes specific to said plurality of said blocked DNA fragments is one denatured DNA probe per blocked DNA fragment or a plurality of denatured DNA probes per blocked DNA fragment.

18. The method according to claim 1, wherein said target DNA sequences to be sequenced have an average sequence length between 10 and 10,000 base pairs.

19. The method according to claim 1 for identifying a treatment based on said tumor characterization.

20. The method according to claim 1, further comprising tumor mapping wherein the genetic aberrations of the tumor are shown in relation to recommended treatments for the tumors characterized by the genetic aberrations, said recommended treatments being obtained by comparing genetic aberrations of the tumor analyzed with genetic aberrations of reference tumors and their reference therapeutic treatments recorded in a database.

21. An agent or agents for treating a tumor in a patient whose tumor has been characterized by the method according to claim 1, said agent or agents being selected from the group comprising PARP inhibitors, targeted therapies, immunotherapy, chemotherapy, radiotherapy, DNA-damaging agents such as platinum-based agents, adjuvant therapies, tyrosine kinase inhibitors, immune checkpoint inhibitors, erlotinib, gefitinib, afatinib, osimertinib, dacomitinib, cabozantinib, crizotinib, alectinib, ceritinib, brigatinib, vemurafenib, encorafenib, dabrafenib, trametinib, cobimetinib, docetaxel, paclitaxel, gemcitabine, pemetrexed, pembrolizumab, atezolizumab, nivolumab, durvalumab, olaparib, niraparib, talazoparib, rucaparib, trastuzumab, pertuzumab, neratinib, a treatment or therapy approved or being developed.

22. A method using a plurality of double-stranded DNA hybridization probes to carry out targeted sequencing of DNA fragments containing simple sequences and DNA fragments containing complex sequences from a sample from a cancer patient, said sample comprising at least one tumor cell.

23. A method for treating cancer in a patient comprising the steps of a) characterizing the tumor of a patient, b) determining a treatment or treatments to be administered comprising one or more agents selected from the group comprising PARP inhibitors, targeted therapies, immunotherapy, chemotherapy, radiotherapy, DNA-damaging agents such as platinum-based agents, adjuvant therapies, tyrosine kinase inhibitors, immune checkpoint inhibitors, erlotinib, gefitinib, afatinib, osimertinib, dacomitinib, cabozantinib, crizotinib, alectinib, ceritinib, brigatinib, vemurafenib, encorafenib, dabrafenib, trametinib, cobimetinib, docetaxel, paclitaxel, gemcitabine, pemetrexed, pembrolizumab, atezolizumab, nivolumab, durvalumab, olaparib, niraparib, talazoparib, rucaparib, trastuzumab, pertuzumab, neratinib, a treatment or therapy approved or being developed, and c) administering the treatment or treatments to said patient, wherein tumor characterization is implemented by applying the method according to claim 1.

24. The method according to claim 23, comprising the step of repeating the tumor characterization of said patient over time and determining whether another treatment should be administered to said patient.

25. A theranostic report of a cancer patient, obtained by implementing the method according to claim 1, for determining a treatment or therapy approved or being developed for the cancer of the patient.
